(19) United States
(12) Patent Application Publication
(10) Pub. No.: US 2016/0067155 A1
(43) Pub. Date: Mar. 10, 2016

(54) FOAMABLE COSMETIC PRODUCT

(71) Applicants: Tadahiro Shimada, Zushi-shi (JP); Hiroko Shimada, Zushi-shi (JP); Hanae Shimada, Zushi-shi (JP); Yo Nakajima, Muroto-shi (JP)

(72) Inventors: Tadahiro Shimada, Zushi-shi (JP); Hiroko Shimada, Zushi-shi (JP); Hanae Shimada, Zushi-shi (JP)

(21) Appl. No.: 14/849,838

(22) Filed: Sep. 10, 2015

(30) Foreign Application Priority Data
Sep. 10, 2014 (JP) 2014-184348

(51) Int. Cl. A61K 8/04; A61K 8/365; A61K 8/34; B65D 81/32; A61Q 19/00; A61Q 5/02; A61Q 19/10; A61K 8/19; A61K 8/60

(52) U.S. Cl. CPC A61K 8/046; A61K 8/19; A61K 8/365; A61K 8/345; A61K 8/602; A61Q 19/00; A61Q 5/02; A61Q 19/10; B65D 81/32

(57) ABSTRACT

Provided is a cosmetic product which can generate a carbon dioxide gas to cause foaming by a simple method and having excellent usability. Provided is a cosmetic product comprising a first composition and a second composition different from the first composition as constituent elements of the cosmetic product and which foams by mixing the first composition and the second composition, wherein the first composition is a liquid or creamy composition containing a bicarbonate and/or a carbonate, and the second composition is a liquid or creamy composition containing an α-hydroxy acid and/or a saturated dibasic acid in which the content of water is 1.0-fold or less in terms of weight ratio with respect to the α-hydroxy acid and/or the saturated dibasic acid.

### BACKGROUND OF THE INVENTION

1. Field of the Invention

The present invention relates to a foamable cosmetic product which is used by mixing a plurality of compositions to generate a carbon dioxide gas.

2. Description of the Related Art

Conventionally, an effect of a carbon dioxide gas on a living body has attracted attention. By way of percutaneous absorption, a carbon dioxide gas dilates blood vessels to promote blood circulation and binds to hemoglobin to liberate oxygen, thereby stimulating cell activity (cell activity by the Bohr effect).

Recently, various such cosmetic products utilizing a carbon dioxide gas are commercially marketed and gain popularity. Specifically, it is suggested that in use, a plurality of compositions are mixed, a carbon dioxide gas is generated by a chemical reaction thereof, to give a foamy cosmetic product which is applied to skin.

In most cases, however, conventional foamable cosmetic products include 1) those in which at least one of a composition containing a carbonate and a composition containing an acid is prepared in the form of a powder or granule, and mixed with a liquid base in use (Japanese Patent No. 4912492 and the like), and 2) those in which a carbon dioxide gas is previously charged in an accumulating aerosol vessel or the like and, in use, discharged and mixed with other cosmetic base.

### SUMMARY OF THE INVENTION

Of the above-described conventional foamable cosmetic products, the above-described form 1) had a problem that a powder or granule should be taken out and dissolved in a liquid base in use, thus, the mixing work is troublesome. Particularly, when used in prolonged massage or the like in the situation of esthetic care and the like, the mixing work as described above has to be conducted in each case, impracticably.

The above-described form 2) had a problem that if labor for previously filling a carbon dioxide gas in a vessel and cost for a spray apparatus are taken into consideration, the usable case is limited. That is, the form 2) can be used only in limited commercial facilities and is not suitable for use in standard home.

Then, the present invention has an object of providing a cosmetic product capable of generating a carbon dioxide gas and causing foaming by a simpler method and which is excellent in usability.

For solving the above-described problem, the present invention is as described below.

A cosmetic product comprising a first composition and a second composition different from the first composition as constituent elements of the cosmetic product and which foams by mixing the first composition and the second composition, wherein

the first composition is a liquid or creamy composition containing a bicarbonate and/or a carbonate, and

the second composition is a liquid or creamy composition containing an α-hydroxy acid and/or a saturated dibasic acid in which the content of water is 1.0-fold or less in terms of weight ratio with respect to the α-hydroxy acid and/or the saturated dibasic acid.

Since both the first composition and the second composition are in the form of a liquid or cream, such a cosmetic product can be mixed easily and shows extremely excellent usability. Since the content of water in the second composition is not higher than the content of the α-hydroxy acid and/or the saturated dibasic, foam quality and foam retention are excellent.

In a preferred embodiment of the present invention, the content of water in the second composition is 6 wt % or less.

If the content of water in the second composition is in the above-described range, foam quality and foam retention are more excellent. Particularly, since makeup cosmetic products are often oily preparations, when the present invention is applied cleansing cosmetic products, detergency can be enhanced by reducing water.

In a preferred embodiment of the present invention, at least one of the above-described first composition and second composition contains a compound containing a structure selected from polyhydric alcohols, reducing sugars, sugar alcohols and derivatives thereof.

By adopting such an embodiment, foaming and foam retention when the first composition and the second composition are mixed are excellent. Specifically, fine bubbles can be generated sufficiently.

When the first composition is in the form of a liquid or cream, it is necessary that the concentration of a bicarbonate or a carbonate in the composition is limited to certain level or lower from the standpoint of prevention of precipitation, however, also in this case, it is important that these compounds are contained from the standpoint of realizing sufficient foaming and foam retention.

In a preferred embodiment of the present invention, the above-described compound containing a structure selected from polyhydric alcohols, reducing sugars, sugar alcohols and derivatives thereof is a moisturizer and/or a surfactant.

By adopting such an embodiment, it becomes possible to realize foaming and foam retention while enhancing the moisture-retaining property and stability of a cosmetic product. When the present invention is applied to cleansing cosmetic products, a high cleansing effect (makeup removing effect) can be obtained.

In a preferred embodiment of the present invention, the above-described surfactant is saponin.

When saponin is used as the surfactant as described above, at least some of foaming, foam quality, rinsing facility and cleansing effect (makeup removing effect), preferably, all of them are improved.

In a preferred embodiment of the present invention, the total content of the compound containing a structure selected from polyhydric alcohols, reducing sugars, sugar alcohols and derivatives thereof is 30 to 70 wt % with respect to the total amount of the first composition and the second composition.

When the content of the above-described compound is as described above, foaming and foam retention of a cosmetic product can be further improved. When the present invention is applied to cleansing cosmetic products, a high cleansing effect (makeup removing effect) can be obtained.

In a preferred embodiment of the present invention, the above-described second composition contains a polyhydric alcohol.

When the second composition contains a polyhydric alcohol, foam quality and foam retention are improved.

In a preferred embodiment of the present invention, at least a part of the above-described bicarbonate and/or carbonate is coated with a coating agent.

By coating at least apart of the bicarbonate and/or the carbonate with a coating agent as described above, at least some of foaming, foam quality, prolonged foam retention, rinsing facility and cleansing effect (makeup removing effect), preferably, all of them are improved.

The present invention is a packaged cosmetic product obtained by packaging the above-described cosmetic product into a package wherein the above-described package has a first tank and a second tank and discharge portions for discharging the contents of the above-described first tank and second tank, and the above-described first composition is packaged in the first tank and the above-described second composition is packaged in the second tank, mutually separately.

In the case of such a packed cosmetic product, by discharging the first composition and the second composition from the discharge portions, these compositions can be mixed easily.

In a preferred embodiment of the present invention, the discharge amounts from the above-described discharge portions of the first composition and the second composition are such amounts that the α-hydroxy acid and/or the saturated dibasic acid is 1:0.2 to 1:5 in terms of weight ratio with respect to the bicarbonate and/or the carbonate.

By discharging at such amounts, a cosmetic product showing appropriate foaming and foam retention is obtained.

In a preferred embodiment of the present invention, the content of the above-described bicarbonate and/or carbonate in the first composition is such an amount that the content of the bicarbonate and/or the carbonate is 1.0 wt % or more and less than 45 wt % with respect to the total amount of the above-described first composition and second composition when mixed.

The present invention is a method of preparing a cosmetic product comprising mixing a first composition and a second composition to generate a carbon dioxide gas and to cause foaming, wherein

the above-described first composition is a liquid or creamy composition containing a bicarbonate and/or a carbonate, and

the above-described second composition is a liquid or creamy composition containing an α-hydroxy acid and/or a saturated dibasic acid in which the content of water is 1.0-fold or less in terms of weight ratio with respect to the α-hydroxy acid and/or the saturated dibasic acid.

Also in the preparation method of the present invention, as components contained in each composition, preferred embodiments described above can be adopted.

It is preferable to mix the above-described first composition and second composition so that the mixed amount of the α-hydroxy acid and/or the saturated dibasic acid with respect to the bicarbonate and/or the carbonate is 1:0.2 to 1:5 in terms of weight ratio.

### Effect of the Invention

The cosmetic product, the packaged cosmetic product and the method of preparing a cosmetic product of the present invention can be used simply, and enlarge application of foamable cosmetic products utilizing a carbon dioxide gas. Further, the cosmetic product of the present invention is excellent in foam quality and foam retention.

### DESCRIPTION OF PREFERRED EMBODIMENTS

Embodiments for carrying out the invention will be illustrated in detail below.

The cosmetic product of the present invention is a cosmetic product comprising a first composition and a second composition different from the first composition as constituent elements of the cosmetic product and which generates a carbon dioxide gas and foams by mixing the first composition and the second composition.

Each composition will be illustrated below.

### <1> First Composition

The first composition contains a bicarbonate and/or a carbonate.

The bicarbonate includes preferably sodium hydrogen carbonate, ammonium hydrogen carbonate and potassium hydrogen carbonate. The carbonate includes preferably sodium sesquicarbonate, potassium carbonate, sodium carbonate and ammonium carbonate.

These may be used only singly or some of them may be used in combination.

The content of the bicarbonate and/or the carbonate in the first composition is preferably 3 wt % or more, more preferably 5 wt % or more, further preferably 10 wt % or more. The upper limit of the content of the bicarbonate and/or the carbonate can be controlled within the range wherein preparation production is possible. As a rough indication, the upper limit can be preferably set at 80 wt %.

The content of the bicarbonate and/or the carbonate with respect to the first composition can be determined based on the proportion to the total amount when mixed with the second composition described later.

That is, the content of the bicarbonate and/or the carbonate is preferably 0.5 wt % or more and less than 45 wt %, further preferably 5 wt % or more and less than 40 wt % with respect to the total amount of the first composition and the second composition.

It is preferable that at least a part of the bicarbonate and/or the carbonate is coated preferably with a coating agent. The coating agent includes fats, waxes, fatty acids and fatty acid derivatives, higher alcohols, sugars (including polysaccharides and sugar alcohols), natural polymers and derivatives thereof, synthetic and semi-synthetic polymers and the like.

The fats and waxes include ceramics, bees wax, isopropyl linoleate, di(isosteryl/phytosteryl) dimer dilinoleate, hydrogenated olive oil stearyl ester and the like.

The fatty acids and fatty acid derivatives include myristyl trisiloxane, stearic acid, isopropyl stearate and the like.

The high alcohols include behenyl alcohol, lanolin alcohol and the like.

The sugars include sugar alcohols such as mannitol and the like, polysaccharides such as xanthan gum (also is a natural polymer) and the like.

The natural polymers include pullulan, pectin, gelatin, cellulose gum, dextran, gluten, starch, glucosyl trehalose and the like.

The synthetic and semi-synthetic polymers include ethylcellulose, PEG, polyacrylic acid, phthalic acid soy bean protein and the like.

The coating agent is preferably an extended-release coating agent, and specifically, ethylcellulose and the like are preferably mentioned.

Coating with the coating agent can be conducted, for example, by the following method.

The bicarbonate and/or the carbonate is, if necessary, mixed with a filler, to give a material to be coated. Here, the filler includes mineral powders such as talc, kaolin, titanium oxide, silica and the like, or those obtained by adding coloring matters and perfumes to organic powders such as a cellulose powder, a plant body powder and the like.

On the other hand, the coating agent is dissolved in a suitable solvent (for example, ethanol and the like when ethylcellulose is used) (coating agent-dissolved solution).

The above-described material to be coated is sprinkled on the coating agent-dissolved solution, and these are kneaded in the form of a paste. After sufficient kneading, the paste is thinly drawn, and the solvent is volatilized. By volatilizing the solvent, a coated material is prepared. This is pulverized to a particle size of about 10 to 100 μm, and sieved, and a uniform dry powder is taken out.

The use amount of the coating agent can be appropriately adjusted depending on the amount of the bicarbonate and/or the carbonate to be contained. For example, the use amount can be usually 0.001 to 1-fold, preferably 0.01 to 0.5-fold, further preferably 0.02 to 0.1-fold, as a rough indication, with respect to the mass of the bicarbonate and/or the carbonate.

By coating at least a part of the bicarbonate and/or the carbonate with the coating agent, at least some of foaming, foam quality, foam retention, rinsing facility and cleansing effect (makeup removing effect), preferably, all of them are improved.

The first composition preferably contains a compound containing a structure selected from polyhydric alcohols, reducing sugars, sugar alcohols and derivatives thereof. By this, it becomes possible to realize excellent foaming and foam retention.

It is preferable that these compounds function as a moisturizer or a surfactant.

The first composition preferably contains a moisturizer selected from polyhydric alcohols, reducing sugars, sugar alcohols and derivatives thereof. By this, it becomes possible to realize excellent foaming and foam retention.

As the above-described moisturizer, for example, polyglycerin, diglycerin, glycerin, propylene glycol, dipropylene glycol, 1,3-butylene glycol, fructose, glucose, sucrose, lactose, sorbitol, mannitol, inositol, erythritol, xylityl glucoside, trehalose, xylitol, xylose, maltitol, raffinose, glycosyl glucose, maltose, oligosaccharide, glycosyl trehalose and derivatives thereof are preferably used.

The content of these moisturizers is preferably 1 wt % or more, further preferably 2 wt % or more, particularly preferably 5 wt % or more. The upper limit can be determined in the range wherein preparation production is possible, and can be set at 10 wt % as a rough indication.

The first composition preferably contains s surfactant selected from alkylglucoside type nonionic surfactants, ester type nonionic surfactants, alkyl ether type nonionic surfactants and alkyl polysaccharide carboxylic salts.

The above-described surfactant includes polyglycerin fatty acid esters, alkyl maltitol ethers, alkyl maltitol esters, maltitol hydroxyalkyls, sucrose fatty acid esters, methylglycoside fatty acid esters, alkyl glycoside, alkyl polyglycoside, carboxymethyl glucan sodium and the like.

The content of these surfactants is preferably 1 wt % or more, further preferably 2 wt % or more, particularly preferably 5 wt % or more. The upper limit can be determined in the range wherein preparation production is possible, and can be set at 10 wt % as a rough indication.

The total content of the moisturizer and the surfactant described above in the first composition is preferably 2 wt % or more, further preferably 5 wt % or more. The upper limit can be determined in the range wherein preparation production is possible, and can be set at 20 wt % as a rough indication.

In the first composition, it is further advantageous that components capable of reinforcing foam wall formation and increasing the viscosity of a foam solution are blended in addition to the above-described components, and it is preferable that surfactants and thickening agents composed of a polymer compound are contained.

By this, it becomes possible to stabilize foams produced by generation of a carbon dioxide gas, thereby improving foam retention.

As such components, the following components are mentioned.

Natural surfactants composed of elastin, surfactin sodium, hydrogenated lysolecithin, saponin, lysolecithin, casein Na, mukurossi peel extract and the like, or derivatives thereof;

Natural polymers composed of starch, alginates such as sodium alginate and the like, carrageenan, guar gum, quince seed, tamarind seed gum, xanthan gum, locust bean gum, gelatin, albumin, collagen, hyaluronic acid, Sclero gum, gum Arabic, tragacanth gum, agar, quince seed and the like, or derivatives thereof;

Semi-synthetic water-soluble polymers composed of carboxymethyl cellulose, sodium lauroyl hydrolyzed silk, hydrolyzed collagen, hydrolyzed sericin, silk fibroin, atelocollagen, cationized guar gum, or derivatives thereof;

Synthetic water-soluble polymers composed of polyvinyl alcohol, polyacrylic acid derivative, hydroxyethyl cellulose, hydroxypropyl cellulose and the like, or derivatives thereof;

Polysoap obtained by crosslinking alkylphenol with formalin, polyoxyethylene alkyl ether, polyoxypropylene alkyl ether, polyoxyethylene-polyoxypropylene alkyl ether, polyoxypropylene-polyoxyethylene block copolymer, hydroxypropyl methyl cellulose stearoxyl ether, inulin laurylcarbamate and the like, or derivatives thereof.

Of them, saponin is preferably used. Plant extracts such as mukurossi peel extract containing saponin, and the like, can also be preferably used. Mukurossi peel extract can be obtained by treating fruit skin of Sapindus mukurossi with a polar solvent. Since a dried substance thereof is commercially available, this can also be used.

By containing saponin, at least some of foaming, foam quality and rinsing facility, preferably, all of them are improved.

The content of these surfactants is preferably 1 wt % or more, further preferably 2 wt % or more, particularly preferably 5 wt % or more. The upper limit can be determined in the range wherein preparation production is possible, and can be set at 10 wt % as a rough indication. The content of the thickening agent is preferably 0.01 wt % or more, further preferably 0.1 wt % or more, particularly preferably 0.5 wt % or more. The upper limit can be determined in the range wherein preparation production is possible, and can be set at 5 wt % as a rough indication.

The content of saponin is preferably 0.005 mass % or more, further preferably 0.01 mass % or more, 0.05 mass % or more, particularly preferably 0.1 mass % or more.

The content of Mukurossi peel extract is preferably 1 wt % or more, further preferably 2 wt % or more, particularly preferably 5 wt % or more, in terms of dry mass.

The first composition is in the form of a liquid or cream. Here, the cream indicates a general idea widely including those having flowability, and is not limited to those of emulsion type. That is, gel and paste are also included. The first composition preferably contains water. The content of water in the first composition can be, for example, about

30 to 90 wt %, preferably about 40 to 80 wt %, as a general indication. The viscosity of the first composition is preferably 300000 mPa·s or less, further preferably 5000 to 50000 mPa·s or less at 25° C.

The first composition can be produced by mixing the above-described essential components with optional components. For production thereof, ordinary methods for producing cosmetic products may be used.

### <2> Second Composition

The second composition contains an α-hydroxy acid and/or a saturated dibasic acid.

The α-hydroxy acid preferably includes citric acid, glycolic acid, tartaric acid and lactic acid. The saturated dibasic acid preferably includes oxalic acid, malic acid, malonic acid, succinic acid, glutaric acid, adipic acid, pimelic acid, azelaic acid.

These may be used only singly or some of them may be used in combination.

The content of the α-hydroxy acid and/or the saturated dibasic acid with respect to the second composition may be determined in a good balance with the amount of the bicarbonate and the carbonate in the first composition to be mixed, and is preferably 0.3 wt % or more, further preferably 1 wt % or more as a rough indication. The upper limit of the content of the α-hydroxy acid and/or the saturated dibasic acid is preferably 20 wt % or less, further preferably 10 wt % or less as a rough indication.

The content of the α-hydroxy acid and/or the saturated dibasic acid with respect to the second composition can be determined based on the proportion to the total amount when mixed with the first composition described above.

That is, the content of the α-hydroxy acid and/or the saturated dibasic acid is preferably 0.2 wt % or more and less than 35 wt %, further preferably 1 wt % or more and less than 15 wt % with respect to the total amount of the first composition and the second composition.

Based on the total amount when mixed with the first composition described above, the content of the α-hydroxy acid and/or the saturated dibasic acid with respect to the content of the bicarbonate and/or the carbonate is preferably 1:0.2 to 1:5.0, further preferably 1:0.3 to 1:3.0 by weight.

The content of water in the second composition is 1.0-fold or less, preferably 0.5-fold or less, further preferably 0.3-fold or less in terms of weight ratio with respect to the α-hydroxy acid and/or the saturated dibasic acid. Further, the second composition may also be in a no-water containing form.

The content of water in the second composition is preferably 10 wt % or less, more preferably 6 wt % or less.

That is, by decreasing the use amount of water in the second composition, it becomes possible to improve foam quality, foam retention and cleansing effect (makeup removing effect) after mixed with the first composition.

It is also preferable for the second composition to contain a compound having a structure selected from polyhydric alcohols, reducing sugars, sugar alcohols and derivatives thereof. As these compounds, those exemplified for the first composition can be likewise used. These dissolve the above-described acids including typically an α-hydroxy acid.

Particularly, the second composition preferably contains a polyhydric alcohol. The polyhydric alcohol includes preferably sugars such as sorbitol, sucrose, fructose, glucose, maltose, trehalose, xylitol and the like, and glycerins such as glycerin, diglycerin, polyglycerin, alkane diol, polyethylene glycol and the like.

Of the above-described compounds, those functioning as a surfactant can be preferably used in the second composition. As the surfactant, a hydrophilic surfactant or a lipophilic surfactant may be used singly, or both the surfactants may be used, depending on the form of the preparation.

The hydrophilic surfactant includes POE glycerin fatty acid ester, POE sorbitan fatty acid ester, sucrose fatty acid ester, alkyl polyglycoside, alkyl polyglyceryl ether and polyglyderin fatty acid ester.

The lipophilic surfactant preferably includes glycerin fatty acid ester, low mol-added POE glycerin fatty acid ester, sorbitan fatty acid ester, low mol-added POE sorbitan fatty acid ester, low-polymerized polyglycerin fatty acid ester and diglycerin monooleate.

For example, in the case of a cleansing cosmetic product, it is preferable that both a hydrophilic surfactant and a lipophilic surfactant are contained. In this case, the content ratio of the hydrophilic surfactant and the lipophilic surfactant in the above-described second composition is preferably 1:10 to 1:0.5, further preferably 1:6 to 1:1.

The content of the above-described compound containing a structure selected from polyhydric alcohols, reducing sugars, sugar alcohols and derivative thereof in the second composition is preferably 70 wt % or more, further preferably 80 wt % or more, particularly preferably 85 wt % or more in terms of the total content of these components. The upper limit is preferably 95 wt %, further preferably 90 wt %.

That is, by using the above-described polyhydric alcohols and/or surfactants as the main dissolving base for the α-hydroxy acid and/or the saturated dibasic acid in the second composition, it is possible to improve foam quality, foam retention and cleansing effect (makeup removing effect) when later mixed with the first composition, while sufficiently dissolving the α-hydroxy acid and/or the saturated dibasic acid.

Also the second composition is in the form of a liquid or cream. The definition of cream is as described above. The viscosity of the second composition is not particularly restricted, and preferably 50000 to 5000000 mPa·s, more preferably 20000 to 100000 mPa·s at 25° C.

The second composition can be produced by mixing the above-described essential components and optional components. For production thereof, ordinary methods for producing cosmetic products may be used.

In a preferred embodiment, it is preferable that both one functioning as a moisturizer and one functioning as a surfactant are present when the first composition and the second composition are mixed.

Therefore, for example, an embodiment in which the first composition contains the above-described moisturizer and the second composition contains the above-described surfactant, and a reverse embodiment thereof are also preferable.

Of course, a case in which both the compositions contain the above-described moisturizer and surfactant is more preferable since mixing is performed quickly.

The content of the above-described compound containing a structure selected from polyhydric alcohols, reducing sugars, sugar alcohols and derivatives thereof is preferably 30 to 70 wt %, more preferably 40 to 60 wt % with respect to the total amount of the first composition and the second composition.

### <3> Cosmetic Product of the Present Invention

The cosmetic product of the present invention has the first composition and the second composition described above as constituent elements. It is necessary that the first composition and the second composition are in separated situation so as not to contact mutually when preserved.

For such an embodiment, for example, it is preferable to use a package having a first tank and a second tank and discharge portions for discharging the contents of the first tank and the second tank. In this case, the first composition is packaged in the first tank and the second composition is packaged in the second tank, mutually separately.

Thought the form of the discharge portion is not particularly restricted, it is preferable to have a dispenser capable of discharging a constant amount. By this, the first composition and the second composition can be quantified respectively and suitable amounts can be mixed.

The package suitable for the present invention includes (1) push nozzle mode dispenser packages having a spring or a valve inside, (2) nozzle packages in which the bottom surface slides to the upper direction simultaneously with discharge of the content due to pressure reduction (air-less mode dispenser), double packages in which a nitrogen gas or a carbon dioxide gas is filled outside an inner bag accommodating the content and the content is discharged from a nozzle by pressure of the gas, and the like. It is also possible that the pressure in an inner bag is reduced to discharge the content.

It is also preferable that the compositions are mixed around a discharge port and the mixture is discharged via one discharge port. In this case, a cosmetic product starting foaming by mixing of the compositions is discharged, for example, onto a hand.

It is also possible that the compositions are discharged form adjacent separate discharge ports. In this case, foaming starts immediately after discharging compositions onto a hand.

Further, a tube-shaped package, pouch-shaped package, a blister package and the like can also be used. Also in this case, the package has two tanks (accommodation portions), and when the package is opened, the compositions accommodated in the accommodation portions are fed through apertures, and mixed on a hand.

In the case of the pouch-shaped package, an embodiment may also be permissible in which by applying pressure to the package, a wall between two tanks is broken and two compositions are mixed.

It is also permissible that the above-described compositions are filled in separate containers constituting a kit. In this case, the compositions are transferred onto a hand, and mixed with a fingertip with slight force, then, foaming starts.

The cosmetic product of the present invention can be applied to various objects such as a cleansing agent, a face wash, a body washing agent, a skin lotion, a milky lotion, a beauty essence, a cream, a facial mask, a massage agent, a hair grower, a mouthwash, a scalp washing agent, a head hair washing agent and the like.

The cosmetic product of the present invention generates a carbon dioxide gas by mixing the first composition and the second composition. Since both the first composition and the second composition are in the form of a liquid or cream, these can be mixed easily and usability is excellent.

### EXAMPLES

The characteristics of the present invention will be explained in detail by further providing examples. In the following examples, % is by weight unless otherwise stated.

### Examples 1 to 2, Comparative Examples 1 to 2

### Cleansing

For components of the tank A, sodium hydrogen carbonate was dissolved in water, other components were added sequentially, and dissolved uniformly.

For components of the tank B shown in Table 1, components were mixed and dissolved uniformly.

Tank A components and tank B components were filled, respectively, in respective tanks of a two tank-integrated tube.

Subsequently, five estheticians (professional panelist) were allowed to discharge use amounts of the tank A components and the tank B components from the tube and to mix them using fingers. Foam quality was evaluated by them according to the following standard. Points by five panelists were averaged. For foam retention, time from directly after forming foam having a height of 2 cm until the form height became ⅓ was measured. The results regarding respective evaluations are shown in Table 1.

### <Evaluation Standard—Foam Quality>

3: fine foam which is creamy and having elasticity
2: fine foam which is creamy but slightly lacking in elasticity
1: coarse foam which is not creamy and lacking in elasticity

| | | (wt % in tank |
|---|---|---|
| Tank A component | | A component) |
| (1) | water | 71.6 (to 100) |
| (2) | dipropylene glycol | 2.0 |
| (3) | diglycerin | 2.0 |
| (4) | glycosyl trehalose | 3.0 |
| (5) | sodium hydrogen carbonate | 6.0 |
| (6) | xanthan gum | 1.0 |
| (7) | lauryl hydroxyl sulfobetaine liquid (30%) | 10.0 |
| (8) | methyl paraben | 0.1 |
| (9) | diethoxy diglycol cyclohexane 1,4-dicarboxylate | 2.0 |
| (10) | cellulose derivative | 0.3 |
| (11) | polyglycerin-10 oleate | 2.0 |

TABLE 1
Table 1
Component of tank B

| | | | Comparative | Comparative |
|---|---|---|---|---|
| | Example 1 | Example 2 | Example 1 | Example 2 |
| Purified water (total amount) | 0 | 6 | 8 | 30 |
| Organic acid (total amount) | 6 | 6 | 6 | 6 |
| Citric acid | 4 | 4 | 4 | 4 |
| Glycolic acid | 2 | 2 | 2 | 2 |
| Diethoxyl succinate | 2 | 2 | 2 | 2 |
| Polyhydric-alcohol (total amount) | 58.5 | 52.5 | 50.5 | 28.5 |
| 1,2-propanediol | 5 | 5 | 5 | 5 |
| Glycerin | 53.5 | 47.5 | 45.5 | 23.5 |
| Polyglycerin-10 oleate | 15 | 15 | 15 | 15 |
| Polyglycerin-10 distearate | 1.5 | 1.5 | 1.5 | 1.5 |
| Polyglyceryl-3 caprate | 6 | 6 | 6 | 6 |
| Polyglyceryl-5 trioleate | 5 | 5 | 5 | 5 |
| Viscosity (25° C., mPa · s) | 300,000 | 60,000 | 10,000 | 1,000 |
| Foam quality | 3.0 | 2.6 | 1.2 | 1.0 |
| Foam retention | 10 min | 7 min | 2 min | 1 min |

(Unit of formulation: mass %)

As a result, foam quality and foam retention were excellent in examples in which the content of water was 1.0-fold by weight or less of an organic acid.

In contrast, in comparative examples in which the content of water was larger than 1.0-fold by weight of an organic acid, foaming and foam retention were poor in comparison with examples.

By this, it was found that foam quality and foam retention are improved if the content of water is 1.0-fold by weight or less in the second composition (composition of tank B).

### Example 3

### Make Cleansing

| | | (wt % in tank |
|---|---|---|
| Tank A component | | A component) |
| (1) | water | 71.6 (to 100) |
| (2) | dipropylene glycol | 2.0 |
| (3) | diglycerin | 2.0 |
| (4) | glycosyl trehalose | 3.0 |
| (5) | sodium hydrogen carbonate | 6.0 |
| (6) | xanthan gum | 1.0 |
| (7) | lauryl hydroxyl sulfobetaine liquid (30%) | 10.0 |
| (8) | methyl paraben | 0.1 |
| (9) | diethoxy diglycol cyclohexane 1,4-dicarboxylate | 2.0 |
| (10) | cellulose derivative | 0.3 |
| (11) | polyglycerin-10 oleate | 2.0 |

| Tank B component | | (wt % in tank B component) |
|---|---|---|
| (1) | glycerin | 50.5 (to 100) |
| (2) | glycosyl trehalose | 2.0 |
| (3) | sucrose | 2.0 |
| (4) | polyglyceryl-10 oleate | 20.0 |
| (5) | polyglyceryl-5 trioleate | 5.0 |
| (6) | diethoxyethyl succinate | 3.0 |
| (7) | polyglycery-2 caprate | 6.0 |
| (8) | glycolic acid | 2.0 |
| (9) | citric acid | 2.0 |
| (10) | succinic acid | 1.0 |
| (11) | water | 0.5 |

For tank A components, (6) and (10) were dissolved in (1), and (2) to (11) were added sequentially, and dissolved uniformly.

For tank B components, (1) to (11) were added, and dissolved uniformly.

These were filled in a two tank type package in which tank A components and tank B components are not mixed.

When tank A components and tank B components were mixed in equal amount, creamy and fine foam could be formed. When this was used for cleansing, foam was maintained and use feeling thereof was excellent.

### Example 4

### Shampoo

| | | (wt % in tank A |
|---|---|---|
| Tank A component | | component) |
| (1) | water | 53.6 (to 100) |
| (2) | white sugar | 4.0 |
| (3) | glycerin | 3.0 |
| (4) | sodium hydrogen carbonate | 7.0 |
| (5) | TEA laureth sulfate (trade name: Emal 20T | 15.0 |
| | manufactured by Kao Corporation) | |
| (6) | sodium lauryl glycol carboxylate liquid | 4.0 |
| (7) | sodium lauroamphoacetate | 6.0 |
| (8) | cellulose derivative (Sun Rose K: Nippon Paper | 0.8 |
| | Crecia Co., Ltd.) | |
| (9) | methyl paraben | 0.1 |
| (10) | hydrolyzed silk | 1.0 |
| (11) | hydrolyzed keratin | 0.5 |
| (12) | decyl glucoside | 5.0 |

| Tank B component | | (wt % in tank B component) |
|---|---|---|
| (1) | glycerin | 35.6 (to 100) |
| (2) | 1,2-propanediol | 2.0 |
| (3) | glycosyl trehalose | 3.0 |
| (4) | polyglyceryl-4-lauryl ether | 5.0 |
| (5) | citric acid | 3.0 |
| (6) | lactic acid | 2.0 |
| (7) | succinic acid | 1.0 |
| (8) | alginin laureth sulfate | 20.0 |
| (9) | lauryl propyl betaine liquid (30%) | 10.0 |
| (10) | polyglyceryl-10 oleate | 10.0 |
| (11) | polyglyceryl-5 stearate | 2.0 |
| (12) | polyglyceryl-5 trioleate | 3.0 |
| (13) | dicapryl carbonate | 3.0 |
| (14) | hydrolyzed chitin | 0.1 |
| (15) | cocoyl hydrolyzed keratin K | 0.3 |

For tank A components, (8), (10) and (11) were dissolved in (1), and (2) to (12) were added sequentially, and dissolved uniformly.

For tank B components, (1) to (15) were heated at 60° C., and dissolved uniformly.

These were filled in a two tank type package in which tank A components and tank B components are not mixed.

When tank A components and tank B components were mixed in equal amount, creamy and fine foam could be formed. When this was used for shampoo, foam was maintained and use feeling thereof was excellent.

### Example 5

### Carbon Dioxide Foam Facial Mask

| Tank A component | | (wt % in tank A component) |
|---|---|---|
| (1) | water | 80.6 (to 100) |
| (2) | raffinose (trade name: Oligo GGF: | 1.0 |
| | manufactured by Asahi Kasei | |
| | Chemicals Corporation) | |
| (3) | xylitol | 1.0 |
| (4) | sorbitol | 1.0 |
| (5) | glycerin | 3.0 |
| (6) | polyglyceryl-6 caprate | 2.0 |
| (7) | polyglyceryl-10 isostearate | 2.0 |
| (8) | polyvinyl alcohol (Poval EG05) | 1.0 |
| (9) | polyvinyl pyrrolidone | 0.9 |
| (10) | plant extract | 0.2 |
| (11) | sodium hydrogen carbonate | 5.5 |
| (12) | xanthan gum | 0.7 |
| (13) | phenoxyethanol | 0.3 |
| (14) | polyacrylate cross polymer-6 | 0.8 |

| | | (wt % |
|---|---|---|
| Tank B component | | in tank B component) |
| (1) | hydrolyzed starch | 2.0 |
| (2) | glycosyl trehalose | 3.0 |
| (3) | glycerin | 59.55 (to 100) |
| (4) | 1,2-propanediol | 5.0 |
| (5) | polyvinyl pyrrolidone | 1.3 |
| (6) | polyglyceryl-10 diisostearate | 17.0 |
| (7) | polyglyceryl-2 caprate (Sun Soft Q-10DC) | 3.0 |
| (8) | polyglyceryl-5 stearate | 2.0 |
| (9) | stearic acid sucrose ester | 2.0 |
| (10) | Carbomer | 0.3 |
| (11) | water | 0.5 |
| (12) | citric acid | 3.0 |
| (13) | lactic acid | 0.5 |
| (14) | malic acid | 0.5 |
| (15) | perfume | 0.05 |

For tank A components, (8), (12) and (14) were dissolved in (1), and (2) to (13) were added sequentially, and dissolved uniformly.

For tank B components, (10) was wetted in (4), and (1) to (15) were added, and dissolved uniformly at 60° C.

These were filled in a two tank type package in which tank A components and tank B components are not mixed.

When tank A components and tank B components were mixed in equal amount, creamy and fine foam could be formed. When this was used for facial mask, foam was maintained and use feeling thereof was excellent.

### Example 6

### Carbon Dioxide Foam Facial Mask

| | | (wt % in tank A |
|---|---|---|
| Tank A component | | component) |
| (1) | water | 80.5 (to 100) |
| (2) | sodium hyaluronate | 0.1 |
| (3) | maltitol | 3.0 |
| (4) | mozuku extract (trade name:Rim veil PD: JTS) | 2.0 |
| (5) | sodium hydrogen carbonate | 7.0 |
| (6) | polyglyceryl-4-lauryl ether (trade name: Sun | 2.0 |
| | Ether L-4: Taiyo Kagaku Co., Ltd.) | |
| (7) | Sclerotium gum | 2.0 |
| (8) | Royal jelly extract | 0.2 |
| (9) | vitamin B₆ hydrochloride | 0.05 |
| (10) | tranexamic acid | 0.5 |
| (11) | dipotassium glycirrhizinate | 0.05 |
| (12) | methyl paraben | 0.1 |
| (13) | 95% ethanol | 2.0 |
| (14) | capryl glyceryl (trade name: Sun Soft No. 700 | 0.5 |
| | P-2-C) | |

| | | (wt % in tank |
|---|---|---|
| Tank B component | | B component) |
| (1) | glycerin | 82.339 (to 100) |
| (2) | sorbitol (70%) | 5.0 |
| (3) | citric acid | 5.0 |
| (4) | malic acid | 0.2 |
| (5) | ascorbic acid | 0.3 |
| (6) | sucrose stearate | 1.0 |
| (7) | polyglyceryl-10 dioleate | 3.0 |
| (8) | polyglyceryl-10 isostearate | 2.0 |
| (9) | human oligopeptide-1 | 0.001 |
| (10) | Saccharomyces/rice fermentation liquor | 0.2 |
| (11) | Peony root extract | 0.1 |
| (12) | trimethylglycine | 0.3 |
| (13) | polyglyceryl-10 distearate | 0.5 |

For tank A components, (2) and (7) were dissolved in (1), and (3) to (14) were added sequentially, and dissolved uniformly.

For tank B components, (1) to (13) were mixed, and dissolved uniformly.

Tank A components and tank B components were filled separately in a pouch package (packing) divided into two tanks. When tank A components and tank B components were mixed in equal amount, creamy and fine foam could be formed. When this was used for facial mask, foam was maintained and use feeling thereof was excellent.

### Examples 7 to 10

### Cleansing

A cleansing agent was produced excepting that tank A components were changed as described in the following formulation in Cleansing in Example 1 (Example 7). A coating powder was prepared according the method described above.

Further, according to Example 7, one in which the mukurossi peel extract powder (3) was substituted by water (Example 8), one in which ethyl cellulose among constituent components of the coating powder (8) was substituted by water (Example 9), and one in which both the mukurossi peel extract powder (3) and ethyl cellulose among constituent components of the coating powder (8) were substituted by water (Example 10) were also produced.

Tank A component

| | | |
|---|---|---|
| (1) | water | 76.45 (to 100) |
| (2) | sodium hydrogen carbonate | 7.0 |
| (3) | mukurossi peel extract powder | 1.0 |
| | (containing saponin) | |
| (4) | methyl paraben | 0.15 |
| (5) | xanthan gum | 0.5 |
| (6) | Sclerotium | 0.9 |
| (7) | hydrolyzed starch + glycosyl trehalose mixture | 4.0 |
| | (Torunare (trade name)) | |
| (8) | coating power | 10.0 |
| (8-1) | sodium hydrogen carbonate | (60) |
| (8-2) | kaolin | (20) |
| (8-3) | talc | (5) |
| (8-4) | silica | (10) |
| (8-5) | mannitol | (4.599) |
| (8-6) | iron oxide | (0.001) |
| (8-7) | ethyl cellulose | (0.4) |

Tank A components and tank B components were filled, respectively, in respective tanks of a two tank-integrated tube.

For each product, foaming, foam quality, foam retention, rinsing facility and makeup removing effect were evaluated according to the following standards.

Subsequently, five estheticians (professional panelist) were allowed to discharge use amounts of the tank A components and the tank B components from the tube and to mix them using fingers (makeup with oily foundation). Then, foaming, foam quality, rinsing facility and makeup removing effect were evaluated by them according to the following standards. Points by five panelists were averaged. For foam retention, time from directly after forming foam having a height of 2 cm until the form height became ⅓ was measured. The results regarding respective evaluations are shown in Table 2.

### <Evaluation Standard—Foaming>

3: foam is sufficiently formed quickly
2: foam is sufficiently formed but foaming needs a long time
1: foam is not sufficiently formed and foaming needs a long time

### <Evaluation Standard—Foam Quality>

3: fine foam which is creamy and having elasticity
2: fine foam which is creamy but slightly lacking in elasticity
1: coarse foam which is not creamy and lacking in elasticity

### <Evaluation Standard—Rinsing Facility>

3: rinsing facility is obtained (no residual feeling on skin at all)
2: somewhat difficult rinsing (slight residual feeling on skin)
1: difficult rinsing (residual feeling on skin)

### <Evaluation Standard—Makeup Removing Effect>

3: makeup is removed successfully
2: makeup is roughly removed
1: makeup is scarcely removed

TABLE 2
Table 2

| | | | | | Makeup |
|---|---|---|---|---|---|
| | | Foam | | Rinsing | removing |
| | Foaming | quality | Foam retention | facility | effect |
| Example 7 | 3 | 3 | 14 min | 3 | 3 |
| Example 8 | 2.6 | 3 | 12 min | 3 | 2.8 |
| Example 9 | 2.6 | 3 | 10 min | 3 | 2.6 |
| Example 10 | 2.4 | 2.6 | 10 min | 2.6 | 2.6 |

As is understood from Table 2, it was found that foaming, foam quality and rinsing facility are improved by containing saponin. Further, it was found that foaming, foam quality, foam retention, rinsing facility and makeup removing effect are improved by previously coating sodium hydrogen carbonate with a coating agent.

Further, it was found that all of foaming, foam quality, foam retention, rinsing facility and makeup removing effect are improved by combining these constitutions.

1. A cosmetic product comprising a first composition and a second composition different from the first composition as constituent elements of the cosmetic product and which foams by mixing the first composition and the second composition, wherein
the first composition is a liquid or creamy composition containing a bicarbonate and/or a carbonate, and
the second composition is a liquid or creamy composition containing an α-hydroxy acid and/or a saturated dibasic acid in which the content of water is 1.0-fold or less in terms of weight ratio with respect to the α-hydroxy acid and/or the saturated dibasic acid.

2. The cosmetic product according to claim 1, wherein the content of water in the second composition is 6 wt % or less.

3. The cosmetic product according to claim 1, wherein at least one of the first composition and second composition contains a compound containing a structure selected from polyhydric alcohols, reducing sugars, sugar alcohols and derivatives thereof.

4. The cosmetic product according to claim 3, wherein the compound containing a structure selected from polyhydric alcohols, reducing sugars, sugar alcohols and derivatives thereof is a moisturizer and/or a surfactant.

5. The cosmetic product according to claim 4, wherein the surfactant is saponin.

6. The cosmetic product according to claim 3, wherein the total content of the compound containing a structure selected from polyhydric alcohols, reducing sugars, sugar alcohols and derivatives thereof is 30 to 70 wt % with respect to the total amount of the first composition and the second composition.

7. The cosmetic product according to claim 1, wherein the second composition contain a polyhydric alcohol.

8. The cosmetic product according to claim 6, wherein the total content of the compound containing a structure selected from polyhydric alcohols, reducing sugars, sugar alcohols and derivatives thereof in the second composition is 70 wt % or more.

9. The cosmetic product according to claim 1, wherein at least a part of the bicarbonate and/or carbonate is coated with a coating agent.

10. A packaged cosmetic product obtained by packaging the cosmetic product according to claim 1 into a package, wherein
the package has a first tank and a second tank and discharge portions for discharging the contents of the first tank and second tank, and
the first composition is packaged in the first tank and said second composition is packaged in the second tank, mutually separately.

11. The packaged cosmetic product according to claim 10, wherein the discharge amounts from the discharge portions of the first composition and second composition are such amounts that the weight ratio of the α-hydroxy acid and/or the saturated dibasic acid with respect to the bicarbonate and/or the carbonate is in a rage of 1:0.2 to 1:5.0.

12. The packaged cosmetic product according to claim 10, wherein the content of the bicarbonate and/or the carbonate in the first composition is such an amount that the content of the bicarbonate and/or the carbonate is 1.0 wt % or more and less than 20 wt % with respect to the total amount of said first composition and second composition when mixed.

13. A method of preparing a cosmetic product comprising mixing a first composition and a second composition to generate a carbon dioxide gas and to cause foaming, wherein
the first composition is a liquid or creamy composition containing a bicarbonate and/or a carbonate, and
the second composition is a liquid or creamy composition containing an α-hydroxy acid and/or a saturated dibasic acid in which the content of water is 1.0-fold or less in terms of weight ratio with respect to the α-hydroxy acid and/or the saturated dibasic acid.

14. The method of preparing a cosmetic product according to claim 13, wherein at least one of the first composition and second composition contains a moisturizer selected from sugars and derivatives thereof, and/or, a surfactant selected from sugars and derivatives thereof.

15. The method of preparing a cosmetic product according to claim 13, wherein the first composition and second composition are mixed so that the mixed amount of the α-hydroxy acid and/or the saturated dibasic acid with respect to the bicarbonate and/or the carbonate is 1:0.2 to 1:5.0 in terms of weight ratio.
